(19) United States
(12) Patent Application Publication
(10) Pub. No.: US 2012/0329060 A1
(43) Pub. Date: Dec. 27, 2012

(54) DIAGNOSTIC FOR LUNG CANCER USING MIRNA

(76) Inventors: Jayaprakash Karkera, Radnor, PA (US); Mical Raponi, Berkeley, CA (US)

(21) Appl. No.: 13/583,014

(22) PCT Filed: Mar. 14, 2011
(86) PCT No.: PCT/US11/28304

§ 371 Date: Sep. 6, 2012

Related U.S. Application Data
(60) Provisional application No. 61/315,257, filed on Mar. 18, 2010.

(51) Int. Cl. C12Q 1/68

(52) U.S. Cl. 435/6.12

(57) ABSTRACT

The invention provides a method for diagnosis of lung cancer, in particular, non-small cell lung cancer using circulating levels of miRNA. In a particular embodiment, the ratio of miRNA-21 to miRNA-221 can be used to diagnosis the presence of lung cancer or to monitor the response of a lung cancer patient to treatment.

### PRIOR APPLICATION

This application claims priority to U.S. application No. 61/315,257, filed Mar. 18, 2010, which is entirely incorporated herein by reference.

### BACKGROUND

1. Field of the Invention

The invention relates to a method for testing for the presence of lung cancer in a human subject using a miRNA species as the reporter and a different a miRNA as the control.

2. Discussion of the Field

Diagnostic and prognostic assays are standard tools for use by medical professionals and laypersons for the determination of a physiological change in a body cells or tissues which are indicative of a change in health status. MicroRNAs (miRNA) are small (21-25 nucleotides) non-coding regulatory RNAs that control protein expression at the transcriptional level through various mechanisms. About 700 microRNAs (miRNAs) have been identified in the human genome, and more than one-third of all human genes are believed to be regulated by miRNAs. As a single miRNA can regulate entire networks of genes, these new molecules are considered the master regulators of the genome. The cellular content of a miRNA species varies with stage of differentiation, cell type, cell function, and disease. Dysregulation of miRNAs can change or alter tumor suppressor proteins or activate oncogenes. Previous studies have shown that circulating miRNA can be utilized as a tool to gain a better understanding of both benign and malignant tumor conditions.

As certain miRNA have been associated with oncogenesis and relate to a set of proteins known to be associated with cancer, or subsets of cancers such as non-small cell lung cancer, a diagnostic assay based on miRNA could be a streamlined method of detecting the likelihood of up- or down regulation of numerous cancer-related genes.

### SUMMARY OF THE INVENTION

The present invention provides a method for the diagnosis of non-small cell lung cancer (NSCLC) in a human subject, the method comprising: determining the concentration of a first serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the first miRNA is known to be a NSCLC-specific miRNA; determining the concentration of a second serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the second miRNA is known to be a NSCLC-specific miRNA; and determining the ratio of the first miRNA to the second miRNA whereby the ratio is indicative of a patient having or not having non small cell lung cancer.

### BRIEF DESCRIPTION OF THE DRAWING

FIG. 1 shows the AUC for the derivation and validation data sets.

FIG. 2 shows box plots of the healthy vs. lung cancer patients for miRNA-21:miRNA-221 ratios for individual in the derivation set (A) and the validation set (B) where the cutoff value between the two groups was set at 1.4.

### DETAILED DESCRIPTION OF THE INVENTION

### Definitions & Explanation of Terminology

By “microRNA”, “miRNA”, or “miR-s” is meant a small (19-24 nt), non-protein-coding, endogenous RNA molecule. miRNA genes are estimated to account up to 2-5% of human genes and regulate ˜30% of coding genes. miRNA are located within introns of protein-coding or non-coding genes, in exons of non-coding genes, or in 3 UTRs. miRNA have been shown to regulate mRNA expression and prevent protein production through the RNAi (RNA interference) pathway. Specific sequences of miRNA have been discovered in humans and nonhuman animals, including invertebrates. Specific sequences are denoted by numbers. Disclosure of a large number of miRNAs can be found in, for example, WO03/029459A2 (Max Planck Institute). A searchable database of published miRNA sequences and annotation is also available on the internet. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download. At present, the miRBase is hosted and maintained in the Faculty of Life Sciences at the University of Manchester, and was previously hosted and supported by the Wellcome Trust Sanger Institute.

For the evaluation of a diagnostic test, predictive values help interpret the results of tests in the clinical setting. The diagnostic value of a procedure is defined by its sensitivity, specificity, predictive value and efficiency. Any test method will produce True Positive (TP), False Negative (FN), False Positive (FP), and True Negative (TN). The “sensitivity” of a test is the percentage of all patients with disease present or that do respond who have a positive test or (TP/TP+FN)×100%. The “specificity” of a test is the percentage of all patients without disease or who do not respond, who have a negative test or (TN/FP+TN)×100%. The “predictive value” or “PV” of a test is a measure (%) of the times that the value (positive or negative) is the true value, i.e., the percent of all positive tests that are true positives is the Positive Predictive Value (PV+) or (TP/TP+FP) x100%. The “negative predictive value” (PV) is the percentage of patients with a negative test who will not respond or (TN/FN+TN)×100%. The “accuracy” or “efficiency” of a test is the percentage of the times that the test give the correct answer compared to the total number of tests or (TP+TN/TP+TN+FP+FN)×100%. The “error rate” calculates from those patients predicted to respond who did not and those patients who responded that were not predicted to respond or (FP+FN/TP+TN+FP+FN)×100%. The overall test “specificity” is a measure of the accuracy of the sensitivity and specificity of a test do not change as the overall likelihood of disease changes in a population, the predictive value does change. The PV changes with a physician's clinical assessment of the presence or absence of disease or presence or absence of clinical response in a given patient.

A predetermined “cutoff value” is specific for the algorithm and parameters related to patient sampling and treatment conditions. When numerical values, scores, such as the ratio of a first miR and a second miR from an individual test subject falls below the cutoff value, the subject is classified as falling into a one group or category, e.g. healthy, and when the numerical value, score, or ratio falls above the cutoff, the subject is classified in the alternative group or category.

### OVERVIEW

The goal of was to determine if the presence of circulating miRNAs from NSCLC patients can serve as non-invasive diagnostic biomarkers of disease and therapy monitoring. As certain miRNA can be upregulated in cancer and therefore act as oncomers, and other miRNA which act as tumor suppressors or act on genes that have tumor suppressor activity, the possibility that both up-regulation and down-regulation of miRNA can be detected in NSCLC patients concurrently raised the possibility of using multiple markers in a diagnostic assay.

The expression of 16 NSCLC-specific miRNAs (miR-146b, miR-155, miR-205, miR-21, miR-221, miR-199a-5p, miR-222, miR-320, miR-223, miR-25, miR-16, miR-191, miR-20a, miR-24, miR-145, and miR-152) was evaluated in 46 NSCLC serum samples ranging from stage Ito IV, including 20 adenocarcinoma and 26 squamous cell carcinoma samples in an initial derivation set of samples along with eighteen healthy controls for comparison.

RNA was extracted from serum using the single-step Trizol method and quantitated using real-time PCR. Of the 16 miRNAs tested, six miRNAs were significantly upregulated in the NSCLC when compared to the normals (p<0.0001 with AUROCs of 0.80 to 0.93). Top Scoring Pair analyses identified the ratio of miR-21 to miR-221 as the most robust predictor of NSCLC status compared to healthy subjects with an AUROC of 0.95 (95% CI; 0.91-0.99, p<0.001) and 17% error rate (leave-one-out cross validation). This miRNA pair was then validated in an independent set of 19 NSCLC and 26 normal serum samples with overall accuracy of 80% (89% and 73% for NSCLC and normal, respectively) and AUC of 0.88 (95% CI: 0.78-0.98). These findings demonstrate the potential of using miR-21 and miR-221 in detecting NSCLC from the serum. Furthermore, these results lead to the development of non-invasive biomarkers for early diagnosis, prognosis, and therapy monitoring in NSCLC.

### Instruments, Reagents and Kits for Performing the Analysis

In one embodiment, the invention provides a system for quantitating miRNA in a patient's sample, comprising an miRNA extraction reagent, one or more miRNA quantitation methods, reagents specific for the miRNA desired to be quantitated, and a means for determining the ratio between selected specific miRNA. The supply of reagents and performance of the operation of the system can be provided as a service for a fee from a vendor. Such a vendor may use validated equipment and practices as mandated and monitored by a government agency for such matters.

### Methods of Using the Invention

miRNA are present and stable in cell-free body fluids including plasma and serum. Circulating miRNA has been shown to gain an information about benign and malignant conditions. Thus, in one method of the invention, the serum collected from a blood sample drawn from a human subject can be used in the method of the invention.

### Clinical Assessment Methods

The use of the chest x-ray as a screening tool include availability and ease of performing the test, low cost, and low risk to the patient. The disadvantages are low sensitivity and specificity. Computed tomography is much more sensitive for detecting small nodules in the lungs that are likely to represent earlier stages of lung cancer. CT screening trials have shown that chest radiographs miss 60% to 80% of the lung cancers detected by CT but are more costly and deliver higher amounts of radiation to the patient. There is also a greater risk of over diagnosis, not only of nonmalignant lung nodules, but other incidental findings as well, which may be found in the course of screening with CT.

Lung cancer is diagnosis relies on radiological findings and histological analysis of biopsy tissue. Lung cancer staging is determined using several criteria. The vast majority of lung cancers are carcinomas, malignancies that arise from epithelial cells. Based on histological criteria, there are two main types of lung carcinoma, categorized by the size and appearance of the malignant cells: non-small cell (80.4%) and small-cell (16.8%) lung carcinoma. The non-small cell lung carcinomas are grouped together because their prognosis and management are similar. There are three main sub-types: squamous cell lung carcinoma, adenocarcinoma, and large cell lung carcinoma.

Currently, the most widely recognized and utilized lung cancer classification system is the 4th revision of the Histological Typing of Lung and Pleural Tumours, published in 2004 as a cooperative effort by the World Health Organization and the International Association for the Study of Lung Cancer. It recognizes numerous other distinct histopathological entities of non-small cell lung carcinoma, organized into several additional subtypes, including sarcomatoid carcinoma, salivary gland tumors, carcinoid tumor, and adenosquamous carcinoma. The latter subtype includes tumors containing at least 10% each of adenocarcinoma and squamous cell carcinoma. When a tumor is found to contain a mixture of both small cell carcinoma and non-small cell carcinoma, it is classified as a variant of small cell carcinoma and called a combined small cell carcinoma.

Non-small cell lung carcinoma is staged from IA (“one A”; best prognosis) to IV (“four”; worst prognosis). Small cell lung carcinoma is classified as limited stage if it is confined to one half of the chest and within the scope of a single radiotherapy field; otherwise, it is extensive stage. Lung cancer is staged based on the extent and size of the tumor (T), lymph nodes (N) involved, and presence of metastases (M). See, for example, Lung Cancer: a Handbook for staging, imaging, and lymph node classification. By Clifton F. Mountain, M D, Herman I. Libshitz, M D, and Kay E. Hermes, Copyright 1999-2003 by CF Mountain and HI Libshitz, Houston, Tex.

Having described the invention in general terms, specific non-limiting examples are provided below.

### Example 1

### Development of the Test Parameters

In order to determine, the feasibility of using miRNA as a diagnostic marker of lung cancer, miRNA from serum of normal healthy volunteers was compared to that from serum of patients clinically diagnosed with lung cancer. The training set (derivation set) included 18 normal and 48 NSCLC (adenocarcinoma and squamous) and the validation (test) set included 26 normal and 19 NSCLC (adenocarcinoma and squamous).

| | Derivation Set | |
|---|---|---|
| | No. of samples | Validation Set 1 |
| Sample Characteristics | (n = 66) | No. of samples (n = 45) |
| NSCLC Histology | | |
| Adenocarcinoma | 21 (44%) | 9 (47%) |
| Squamous Cell Carcinoma | 26 (54%) | 10 (53%) |
| NSCLC undefined | 1 (2%) | |
| Tumor Stage | | |
| I | 27 (56%) | 15 (79%) |
| II | 12 (25%) | 2 (11%) |
| III | 7 (15%) | 2 (11%) |
| IV | 1 (1%) | |

Serum Isolation from Donor Blood

Blood was collected from healthy donors in Serum separator tubes and allowed to coagulate at room temperature for 1 hour. The blood was then centrifuged at 1500 rpm for 10 min The serum was removed and then a second centrifugation was completed at 10,000 rpm for 10 min to ensure complete removal of cellular debris.

Total RNA isolated using TRIZOL method and Total RNA, containing small RNA, was collected from 250u1 of serum using Trizol LS reagent (Invitrogen Life Technologies) according to manufacturer's protocol. The isolated RNA pellet was resuspended in 80 ul of DEPC-treated water and 5u1 of RNA was used for quantitative real-time PCR (qRT-PCR) assay according to the manufacturer's protocols (TAQMAN® miRNA Assays, Applied Biosystems).

miRNA Analysis

Both the RT reaction and real time PCR were performed according to manufacturer's instructions. The miRNA species screened included: mir-146b, mir-155, mir-205, mir-21, mir-221, mir-223, mir-25, mir-16*, mir-191, mir-20a, mir-24, mir-145, mir-152, mir-199a-5p, mir-222, and mir-320. The concentrations of serum miRNA are given as Ct values (Cycle threshold). The Ct value is defined as the PCR cycle number at which the sample's fluorescence is greater than the threshold. The threshold is set at a value that is based on the variability of the baseline fluorescence. The Ct value assigned to a particular sample reflects the point during the reaction at which a sufficient number of amplicons have accumulated, in that well, to be at a statistically significant point above the baseline. A higher Ct value correlates with lower gene or miRNA expression and vice versa. The Ct values were determined using a threshold setting of 0.2 with an automatic baseline and the mean Ct for duplicate reactions was used for subsequent analysis. All Ct values >40 were replaced with 40 and were disregarded as representing below detectable level of analyte or nonspecific amplification.

### Data Analysis

The data were analyzed SAS 9.1. miRNA selection Criteria was FC>=2 or <=−2, p-value <0.05. Student t-test was used to evaluate differences in miRNA expression between NSCLS and normal. For classification model construction, R package tspair (version 2.9.2) was used to find top scoring pair (TSP) classifiers. All 16 miRNAs were found in all samples and TSP classifiers were identified. Leave-one-out cross-validation (LOOCV) was performed in the derivation data set. TSP model was further tuned in a separate data set and a cut-off value was derived to differentiate diseased vs healthy classification. The third data set was served as validation set for the miR-21:miR-221 pair only.

The score denotes the difference between the probability of observing expression levels of marker-i less than marker-j in two classes. For each pair of markers (i,j) the score can be represented by following: Score=Δij=|pij(C1)−pij(C2)| Where pij(Cm)=Prob(Ri>Rj|Y=Cm) and m={i,j}. Pairs with high scores are viewed as most informative for classification. Wilcox-Man Whitney test was used to compare the ratios of miRNA pairs between two classes.

### Results

For each donor, RNA was prepared on three separate days and miRNA analyzed as described. Four miRNAs were evaluated in two different healthy donors to determine reproducibility of the RNA preparation and qRT-PCR analysis (Table 2).

TABLE 2

| | | miR-21 | miR-221 | miR-16 | miR-155 |
|---|---|---|---|---|---|
| Donor 1 | | | | | |
| RNA Prep 1 | Test 1 | 28.21318 | 29.42379 | 24.3875 | 31.12233 |
| RNA Prep 1 | Test 2 | 28.85366 | 29.57969 | 24.41979 | 30.71934 |
| RNA Prep 2 | Test 1 | 29.2779 | 29.27393 | 24.65336 | 31.40225 |
| RNA Prep 2 | Test 2 | 28.98011 | 29.34603 | 24.55345 | 31.52233 |
| RNA Prep 3 | Test 1 | 29.96791 | 30.4746 | 25.45509 | 32.61868 |
| RNA Prep 3 | Test 2 | 29.92765 | 30.36273 | 25.40787 | 32.43663 |
| Donor 2 | | | | | |
| RNA Prep 1 | Test 1 | 30.22812 | 30.14411 | 26.66854 | 36.18246 |
| RNA Prep 1 | Test 2 | 30.16136 | 30.31006 | 26.90018 | 35.98588 |
| RNA Prep 2 | Test 1 | 30.42692 | 29.93382 | 26.14397 | 35.19082 |
| RNA Prep 2 | Test 2 | 30.26502 | 29.92957 | 26.21709 | 34.9791 |
| RNA Prep 3 | Test 1 | 29.49947 | 30.24351 | 25.68125 | 35.03284 |
| RNA Prep 3 | Test 2 | 29.45856 | 30.32619 | 25.73887 | 35.80497 |

The mean intra-assay CV(%) corresponds to the mean CV for duplicate qRT-PCR reactions of triplicate RNA preparations is shown in the table below (Table 3). The mean intra-assay CV (coefficient of variance) for all 4 miRNAs tested was 0.313% (donor 1) and 0.268% (donor 2). The mean inter-assay CV was 2.30% (donor 1) and 1.45% (donor 2). These values indicate that both the RNA isolation and qRT-PCR-based quantification techniques were reproducible and therefore validate the methods used in this study.

| | |
|---|---|
| Mean intra-assay | |
| CV (%)* | Inter-assay CV (%) |

| | Donor | Donor | Donor | Donor |
|---|---|---|---|---|
| RNA | Sample 1 | Sample 2 | Sample 1 | Sample 2 |
| miR-21 | 0.567 | 0.149 | 2.432 | 1.522 |
| miR-221 | 0.190 | 0.139 | 1.992 | 0.701 |
| miR-16 | 0.121 | 0.228 | 2.196 | 2.282 |
| miR-155 | 0.374 | 0.555 | 2.584 | 1.283 |
| Mean | 0.313 | 0.268 | 2.301 | 1.447 |

For NSCLC, expression of mir-221 is higher than mir-21 in normal and was in the reverse order in NSCLC. mir-21 and mir-221 (Score=0.736) was selected with AUC 0.953 (95% CI: 0.91, 0.99) and difference for the median ratio of mir-21 over mir-221 is 2.39 (p<0.001) between normal and NSCLC. LOOCV results: overall accuracy 83%. For validation, mir-21:mir-221 TSP classifier was validated in a separate data set (normal=26, NSCLC=19). A cut-off threshold was selected at 0.69 that gave the sensitivity of 0.94 and specificity of 0.83 for the training data set. Overall accuracy for the testing data set is 80% (89% and 73% for NSCLC and normal respectively) and AUC is 0.88 (95% CI: 0.78-0.98).

The results were stratified for adeno vs squamous cell lung cancer. The expression of mir-320 was high in normal and low in Adeno while the expression of mir-21 is lower in normal and high in Adeno. mir-21 and mir-320 scored top (Score=0.69) with AUC 0.831 and median ratio difference is 2.57 (p<0.001). For squamous, mir-21 and mir-221 scored top (Score=0.791) with AUC 0.955 and median ratio difference is 2.39 (p<0.001).

Analysis of this data set did not identify mirRNA pairs that can discriminate Adeno and Squamous using TSP algorithm. The best pair is mir-155 with mir-205, which scored 0.18 with AUC=0.55, and ratio difference is 1.88 (p=0.574).

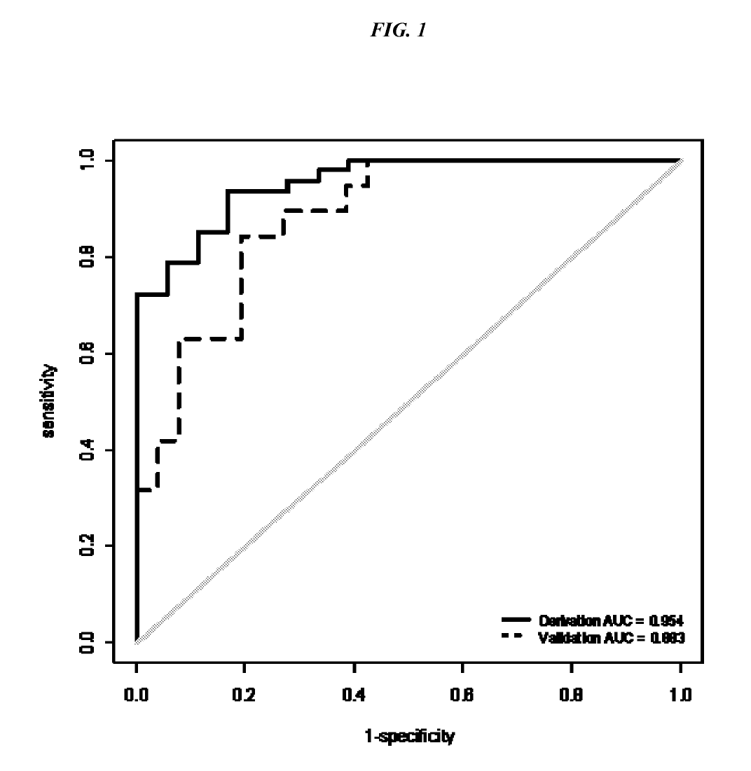

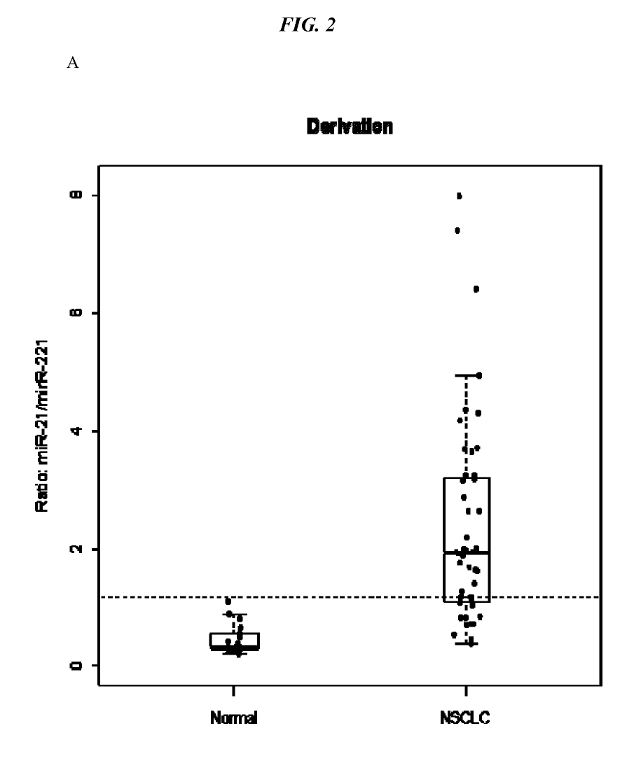

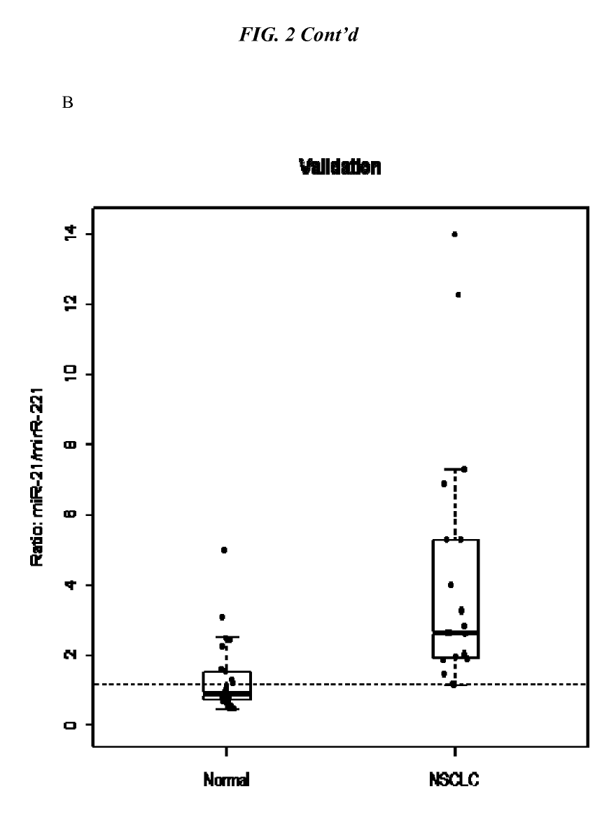

In the model derivation data set, miR-21 and miR-221 were identified as the top scoring pair (Score=0.736) with AUC 0.953 (95% CI: 0.91, 0.99). The second data set was used to derive the classification cut-off. In this validation data set, mir21 and mir221 yield AUC 0.88 (95% CI: 0.78, 0.98). Then the classifier was applied on the Michigan data set and Areas under receiver operating characteristic curves are presented in the FIG. 1 for the two training set and the testing data sets. FIGS. 2A and B presents mir-21:mir-221 ratio for the derivation and validation data sets with optimal cut-off, respectively. For the validation data set, the TSP classifier achieved AUC 0.82 (95% CI: 0.71, 0.82). Using the cut off derived from second data set, 112 out of 126 NSCLC and 8 out of 14 normal in the set 3 is correctly classified with overall 86% accuracy (95% CI: 0.79, 0.91, sensitivity: 0.89 (95% CI: 0.82, 0.94), specificity: 0.57 (95% CI: 0.29, 0.82)). PPV: 0.95 (95% CI: 0.89, 0.98), NPV: 0.36 (0.17, 0.59). Table 2 presents the prediction results in the validation data set.

FIG. 1 shows the AUC for the derivation and validation data sets for the miR-21:miR-221 pair. In the derivation data set, mir21 and mir221 are identified as the top scoring pair (Score=0.736) with AUC 0.953 (95% CI: 0.91, 0.99). In the validation data set, mir21 and mir221 yield AUC 0.88 (95% CI: 0.78, 0.98). From this analysis, the cutoff value was set at 1.4.

FIG. 2A-B show box plots of miR-21:miR-221 ratio in NSCLC and healthy donors for A) the derivation data (test) set of healthy donors (n=18), NSCLC patient samples (n=48); and B) the validation data set of healthy donors (n=26) and NSCLC patient samples (n=19).

### SUMMARY

A robust methodology to study miRNAs in the serum was developed using serum derived miRNA, quantitative PCR, and the R package tspair test (Top Scoring Pair) to identify TSP classifiers. Among the miRNA analyzed in this test and validation data set, miR-21:miR-221 was identified as the most robust predictor of NSCLC status compared to the normals (p<0.0001 with AUROC of 95%).

The diagnostic accuracy of the miRNA ratio validated in an independent set of 19 NSCLC, 26 healthy normal, and 10 benign lung disease (p<0.0001 with AUROC of 88%). For these results the Sensitivity=88%, Accuracy=73%, PPV=63%, NPV=89%.

| Group | Predicted as Normal | Predicted as NSCLC |
|---|---|---|
| Normal | 16 | 10 |
| NSCLC | 2 | 17 |

The ratio of mir-21:miR-221 in serum has clinical application as a non-invasive biomarker for NSCLC.

### Example 2

### Large Data Set Validation

The serum mir-21:miR-221 classifier was applied to an independent set of 126 NSCLC and 14 normal healthy serum samples. These samples were collected from the University of Michigan Hospital with patient consent and Institutional Review Board approval and contained follow-up clinical information for prognostic analysis.

In the model derivation data set of Example 1, mir21 and mir221 are identified as the top scoring pair (Score=0.736) with AUC 0.953 (95% CI: 0.91, 0.99). The second data set was used to derive the classification cut-off. In this validation data set, mir21 and mir221 yield AUC 0.88 (95% CI: 0.78, 0.98). Then the classifier was applied on the Michigan data set and Areas under receiver operating characteristic curves for the two training set and the testing data sets determined.

Using the cut off derived from second data set, 112 out of 126 NSCLC and 8 out of 14 normal in the set 3 is correctly classified with overall 86% accuracy (95% CI: 0.79, 0.91, sensitivity: 0.89 (95% CI: 0.82, 0.94), specificity: 0.57 (95% CI: 0.29, 0.82)). PPV: 0.95 (95% CI: 0.89, 0.98), NPV: 0.36 (0.17, 0.59).

| Group | Predicted as Normal | Predicted as NSCLC |
|---|---|---|
| Normal | 8 | 6 |
| NSCLC | 14 | 112 |

What is claimed:

1. A method for diagnosing whether a human subject has lung cancer, the method comprising:
determining the concentration of a first serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the first miRNA is known to be a NSCLC-specific miRNA;
determining the concentration of a second serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the second miRNA is known to be a NSCLC-specific miRNA; and determining the ratio of the first miRNA to the second miRNA whereby the ratio is indicative of a patient having or not having lung cancer.

2. The method of claim 1, further comprising comparing the ratio of the serum concentration first miRNA and the second miRNA to a cutoff value determined using a training set of data derived from both healthy donors and subjects diagnosed with NSCLC.

3. The method of claim 2 wherein the first miRNA is miRNA-21 and the second miRNA is miRNA-221.

4. The method of claim 3 wherein the ratio of mir-21 over mir-221 is 1.4 or greater.

5. The method of claim 1 wherein the concentration of miRNA is determined by quantitative RT-PCR.

6. A method for monitoring the response of a patient having the diagnosis of non-small cell lung cancer to a therapeutic treatment, the method comprising: determining the concentration of a first serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the first miRNA is known to be a NSCLC-specific miRNA;
determining the concentration of a second serum miRNA selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152, wherein the second miRNA is known to be a NSCLC-specific miRNA; and
determining the ratio of the first miRNA to the second miRNA before and after therapy whereby the change in the ratio is indicative of a patient responding or not responding to the therapy.

7. The method of claim 6, wherein the first miRNA is miRNA-21 and the second miRNA is miRNA-221.

8. The method of claim 7, wherein the ratio of mir-21 over mir-221 is 1.4 or greater.

9. The method of claim 6, wherein the concentration of miRNA is determined by quantitative RT-PCR.

10. A method of any of claim 1-6, wherein the determining step is performed by a computer-assisted device.

11. A test kit for use in diagnosing whether a subject has NSCLC or predicting whether a patient diagnosed with lung cancer has responded to therapy as assessed by the one or more clinical endpoints, comprising: a preprepared substrate capable of quantitating the presence of one of miRNA in a sample of RNA extracted from the subject's serum, selected from the group consisting of miR-146b, miR-155, miR-205, miR-21, miR-221, miR-199a-5p, miR-222, miR-320, miR-223, miR-25, miR-16, miR-191, miR-20a, miR-24, miR-145, and miR-152 and a second but different miRNA, and a method for determining the ratio of the first and second miRNA so quantitated.
